(19) United States
(12) Patent Application Publication
(10) Pub. No.: US 2011/0184285 A1
(43) Pub. Date: Jul. 28, 2011

(54) ULTRASONIC DIAGNOSIS APPARATUS

(76) Inventors: Han DANLI, Dalian (CN); Yu Xiaofeng, Dalian (CN); Huang Zhen, Dalian (CN); Katsuya Hirakui, Nasushiobara-shi (JP)

(21) Appl. No.: 13/010,354

(22) Filed: Jan. 20, 2011

(30) Foreign Application Priority Data
Jan. 22, 2010 (CN) 201010106540.X

(51) Int. Cl. A61B 8/00

(52) U.S. Cl. 600/437

(57) ABSTRACT

According to one embodiment, an ultrasonic diagnosis apparatus comprises, an image display device, an apparatus main body and a support mechanism, wherein the support mechanism comprises a support mechanism main body and a support arm, the image display device is attached to the support mechanism main body, and supported on the apparatus main body by the support arm, and the support mechanism main body is provided with a rotation mechanism which rotates the image display device in an upward-downward direction, a position selecting mechanism which raises or lowers the image display device, and a rotation block mechanism which blocks such rotation as to recline the image display device to a horizontal position in a case where the image display device is disposed at a position other than an uppermost end position in a movement region where the image display device rises or lowers with respect to the rotation mechanism.

### CROSS-REFERENCE TO RELATED APPLICATIONS

This application is based upon and claims the benefit of priority from Chinese Patent Application No. 201010106540. X, filed Jan. 22, 2010; the entire contents of which are incorporated herein by reference.

### FIELD

Embodiments described herein relate generally to an ultrasonic diagnosis apparatus, and more particularly, it relates to a support structure which can rotate an image display surface of an image display device by 90° in an ultrasonic diagnosis apparatus to recline the device downwardly.

### BACKGROUND

An ultrasonic diagnosis apparatus is an essential device in a medical field, in which as shown in FIG. 1, a subject is scanned with an ultrasonic beam via an ultrasonic probe 200 to transmit/receive the resulting signals, and two-dimensional information obtained by the scanning is displayed as an ultrasonic image on an image display device 100, to observe the subject in real time. As the image display device 100 of such an ultrasonic diagnosis apparatus, a liquid crystal display device usually referred to as LCD is used, and for the purpose of allowing an operator and the subject to observe the image on the image display surface without difficulty from any angle, the LCD is supported sometimes by disposing a movable support mechanism which can swivel in a right-left direction or tilt so as to change a pitch angle, or a movable support mechanism including a universal arm. Such a liquid crystal display device can be supported on a main body 400 or an operation panel 300 of the ultrasonic diagnosis apparatus by the movable support mechanism so that the device can rotate in the right-left direction or an upward-downward direction with respect to the main body 400 or the operation panel 300 of the ultrasonic diagnosis apparatus, or can move in a forward-backward direction or the right-left direction. “The right-left direction” mentioned in the present invention means a direction extending between right and left sides of the liquid crystal display device during diagnosis/inspection, and the upward-downward direction mentioned in the present invention means an upwardly and downwardly extending direction of the liquid crystal display device during the diagnosis/inspection. The passage “rotate in the right-left direction” mentioned in the present invention means to rotate the liquid crystal display device toward the left or right side thereof, and the passage “rotate in the upward-downward direction” means to rotate the liquid crystal display device in the upward or downward direction so as to change the pitch angle thereof.

As a conventional technology suggests a display section support structure of an ultrasonic diagnosis apparatus which comprises a display section supporting arm including a first rotary arm having one end vertically positioned in the upper part of a frame of the ultrasonic diagnosis apparatus by a rotation support portion, a second rotary arm having one end relatively rotatably supported at the other end of the first rotary arm, and a third rotary arm having one end relatively rotatably supported at the other end of the second rotary arm, and in which a display section is vertically connected to the other end of the third rotary arm.

However, in such a support structure of the display section, the display section cannot be rotated in the upward-downward direction, and hence an observation range of an observer is limited, i.e., an image on the display section can be observed merely at a predetermined height position.

Moreover, to solve the above problem, as another conventional technology suggests a display section support structure of an ultrasonic diagnosis apparatus which comprises a display section supporting arm including a lower arm having one end vertically positioned in the upper part of a frame of the ultrasonic diagnosis apparatus by a main shaft, an upper arm having one end supported at the other end of the lower arm so as to be relatively rotatable in a right-left direction and an upward-downward direction by a support shaft, and a joint having one end fixed to the other end of the upper arm and the other end for supporting a display section rotatably in the upward-downward direction. Therefore, according to the structure, the rotation of the display section in the upward-downward direction in a small angular range (less than 90°) can be realized, whereby a clear image can readily be observed irrespective of a height position of an observer, for example, irrespective of a state that the observer sits or stands.

However, the ultrasonic diagnosis apparatus is a small-sized movable medical equipment, and the apparatus is often moved from an inspection room to a hospital ward, a treatment room or the like. When the apparatus is moved, the image display device needs to be reclined so that a forward view is not disturbed, thereby securing the forward view. On the other hand, also while the image display device is not utilized, the device needs to be reclined to protect an image display surface of the device from any dust or scratch. Therefore, in recent years, some apparatuses have been developed in which the image display surface can be rotated by 90° and reclined downwardly, but any of the display section support structures according to the conventional technoligies described above cannot realize this function.

To solve this problem, a display section support structure has presently been developed which is a display section movable support structure capable of reclining an image display device (rotating the device downwardly by about 90°). In such a movable support structure, when a rotation supporting point B is set to a place which is not far from the center of the image display device in the same manner as in a conventional technology as shown in FIGS. 16A and 16B and an image display device 100 is reclined downwardly as shown in FIG. 16A, the lower end of the image display device 100 interferes with a support arm 1b, an operation panel 300 or the like. In consequence, as shown in FIG. 16B, the supporting point B for upward/downward rotation needs to be disposed near the lower end of the image display device. When the supporting point B is moved downwardly, a gravity center position G is noticeably away from the supporting point B. When the gravity center position G of the image display device is noticeably away from the supporting position B, a large moment is generated, and a display section 1 easily falls down owing to its own weight. To prevent this problem, a large frictional force has to be applied to the supporting point B, and in this case, there is a drawback that a large operation force is required to rotate the image display device in the upward-downward direction during the diagnosis/inspection, which makes the use of the device difficult. Moreover, when the supporting point B is moved to the lower end of the LCD, the image display section of the LCD moves accordingly upwardly, and hence the center of image display becomes high. In consequence, there is an inconvenience that a sight line of an operator also rises, and therefore a burden of the operator increases and discomfort is brought to the operator during the observation.

### BRIEF DESCRIPTION OF THE DRAWINGS

FIG. 1 is an outline view of an entire ultrasonic diagnosis apparatus;

FIGS. 2A, 2B, and 2C are perspective views showing an entire support mechanism 1 and a main part of an LCD according to a first embodiment;

FIG. 3 is a perspective view showing a connection structure of an LCD attachment plate 2 and a handle mechanism section 4 according to the first embodiment;

FIG. 4 is a perspective view showing a frame 3 according to the first embodiment;

FIG. 5 is an exploded perspective view of a rotation mechanism section 5 according to the first embodiment;

FIG. 6 is a front sectional view of the rotation mechanism section 5 according to the first embodiment;

FIG. 7A is a perspective view of an entire support mechanism main body 1a in which the LCD according to the first embodiment is positioned at the lowermost end;

FIG. 7B is a perspective view of the entire support mechanism main body 1a in which the LCD according to the first embodiment is positioned at the uppermost end;

FIG. 8 is a perspective view of the support mechanism main body 1a in which a grip portion 43 and a link 42 of the handle mechanism section 4 are removed;

FIGS. 9A and 9B are operation explanatory views schematically explaining a main operation of the handle mechanism section 4;

FIG. 10A is a perspective view of a part of the support mechanism main body 1a;

FIG. 10B is a perspective view schematically showing engagement of control blocks 21 and engagement portions 5121 in a part A of FIG. 10A;

FIGS. 11A, 11B, 11C, and 11D are operation explanatory views showing mutual movement and fitting relation between a stopper 22 and a stop groove 526;

FIGS. 12A and 12B are operation explanatory views in which the LCD is moved by hands;

FIGS. 13A, 13B, 13C, and 13D are operation explanatory views in which the LCD according to the first embodiment is moved upwardly and folded;

FIG. 14A is a perspective view of a support mechanism main body according to a second embodiment;

FIG. 14B is an enlarged perspective view of a part B of FIG. 14A according to the second embodiment;

FIGS. 15A, 15B, 15C, and 15D are operation explanatory views schematically showing an operation when an LCD according to a third embodiment is folded;

FIGS. 16A and 16B are explanatory views schematically showing a support mechanism of an LCD of a conventional technology;

FIGS. 17A, 17B, 17C, and 17D are diagrams showing a lowering speed suppression mechanism according to Example 1;

FIGS. 18A and 18B are diagrams showing the lowering speed suppression mechanism according to Example 1; and

FIGS. 19A, 19B, and 19C are diagrams showing the lowering speed suppression mechanism according to Example 1.

### DETAILED DESCRIPTION

In general, according to one embodiment, an ultrasonic diagnosis apparatus comprises; an image display device, an ultrasonic probe, an operation panel, an apparatus main body and a support mechanism, wherein the support mechanism comprises a support mechanism main body and a support arm, the image display device is attached to the support mechanism main body, and supported on the apparatus main body by the support arm, and the support mechanism main body is provided with: a rotation mechanism which rotates the image display device in an upward-downward direction; a position selecting mechanism which raises or lowers the image display device with respect to the rotation mechanism; and a rotation block mechanism which blocks such rotation as to recline the image display device to a horizontal position in a case where the image display device is disposed at a position other than an uppermost end position in a movement region where the image display device rises or lowers with respect to the rotation mechanism.

### First Embodiment

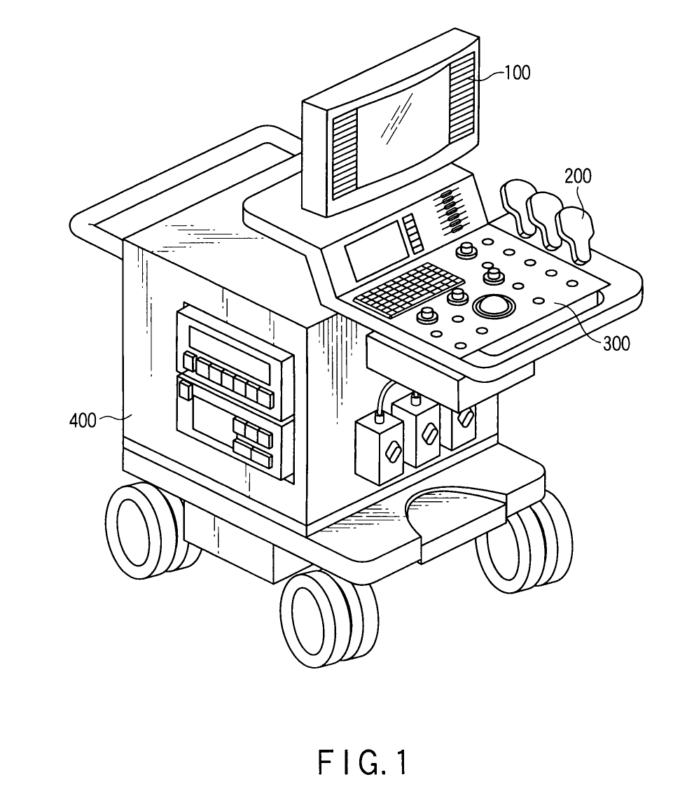

Hereinafter, a main constitution of an ultrasonic diagnosis apparatus according to the present embodiment will schematically be described with reference to FIG. 1. FIG. 1 is an outline view of the whole ultrasonic diagnosis apparatus.

As shown in FIG. 1, the ultrasonic diagnosis apparatus mainly comprises an image display device (an LCD in the present invention) 100, an ultrasonic probe 200, an operation panel 300, an apparatus main body 400 and others. The apparatus main body 400 employs a structure movable by casters attached to the bottom surface thereof, and in the structure, a control device which performs various types of control and processing is mounted.

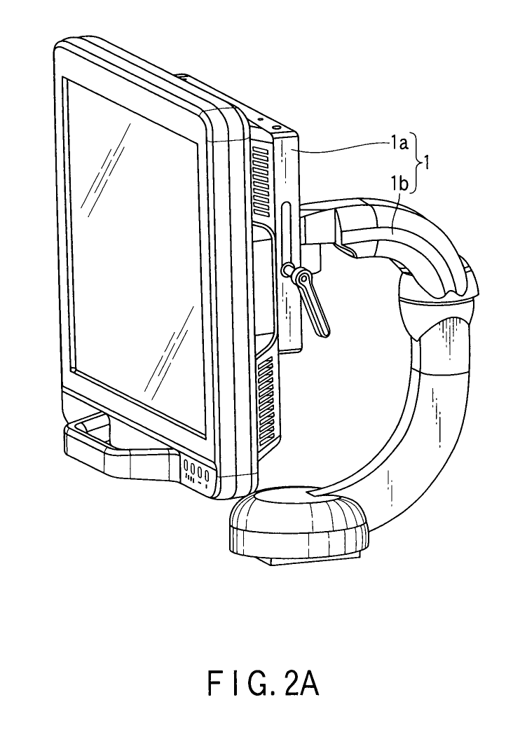

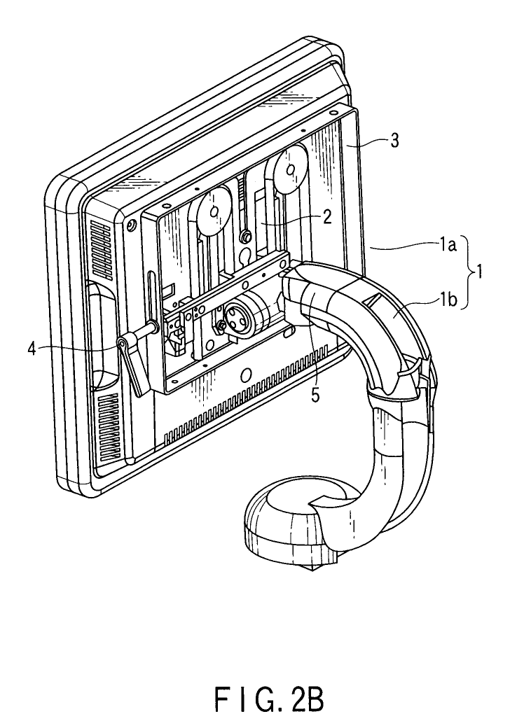

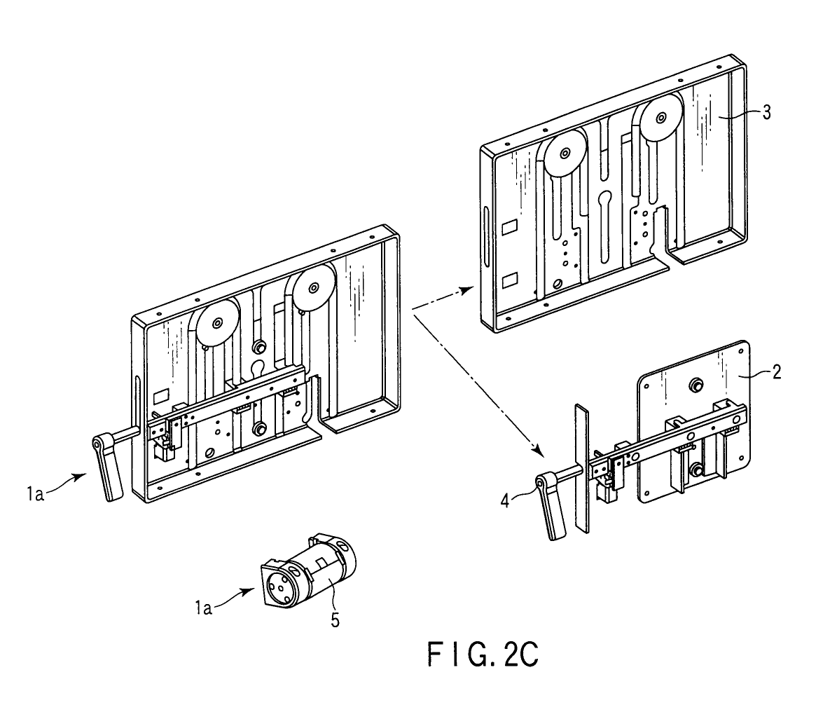

FIGS. 2A and 2B show the appearance of the whole LCD folding (rotating downwardly by about 90°) support mechanism according to the first embodiment. A support mechanism 1 of the LCD 100 comprises a support mechanism main body 1a and a support arm 1b as shown in FIGS. 2A and 2B. The support arm 1b is rotatably attached to the ultrasonic diagnosis apparatus main body 400.

Moreover, as shown in FIG. 2C, the support mechanism main body 1a comprises an LCD attachment plate 2, a frame 3, a handle mechanism section 4, and a rotation mechanism section 5. The LCD is relatively slidably attached to the frame 3 of the support mechanism main body 1a by the LCD attachment plate 2.

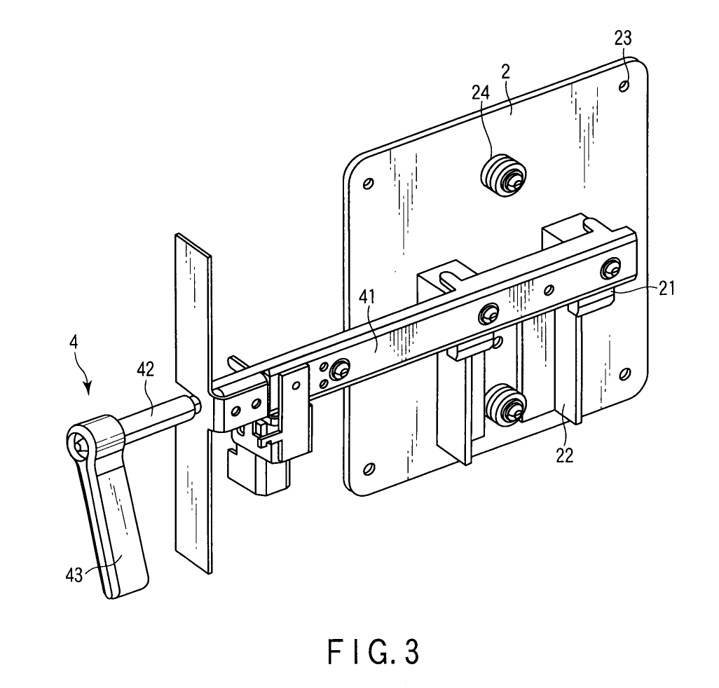

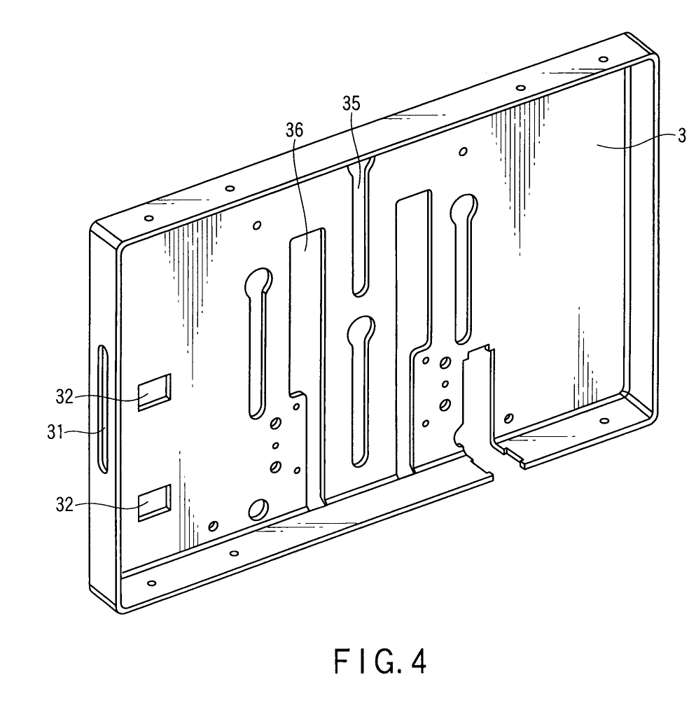

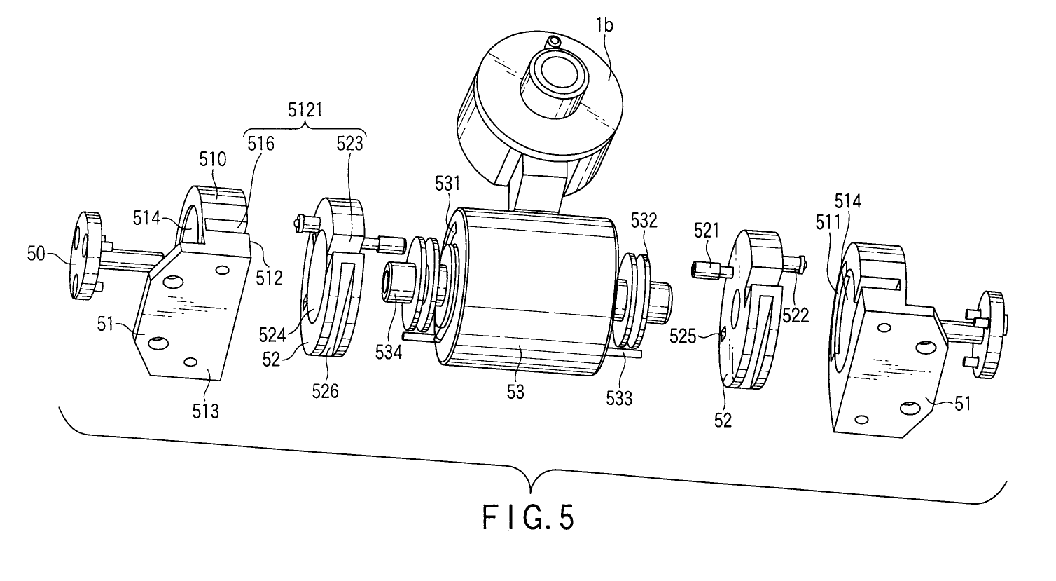

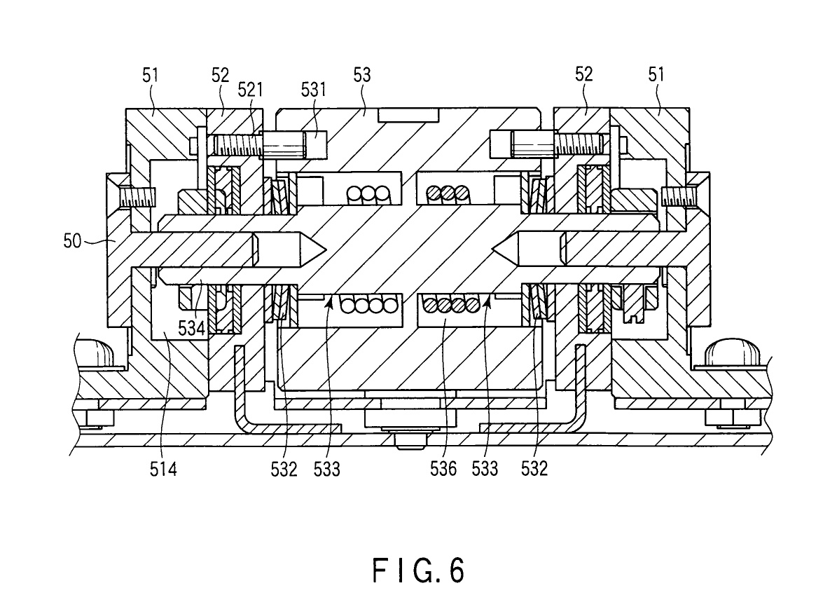

Hereinafter, components constituting the support mechanism main body 1a will be described in detail with reference to FIGS. 3 to 6. FIG. 3 is a perspective view showing a connection structure of the LCD attachment plate 2 and the handle mechanism section 4 according to the first embodiment. FIG. 4 is a perspective view showing the frame 3 according to the first embodiment. FIG. 5 is an exploded perspective view of the rotation mechanism section 5 according to the first embodiment. FIG. 6 is a front sectional view of the rotation mechanism section 5 according to the first embodiment.

As shown in FIG. 3, the LCD attachment plate 2 according to the present invention has a substantially square shape (with the proviso that the shape is not limited to the square shape, and may be a rectangular shape or another), and in the vicinity of the corners of the plate, attachment holes 23 are formed to fix the LCD 100 by fixing means such as screws. Substantially at the center of the LCD attachment plate 2 in an upward-downward direction of the drawing, a pair of control blocks 21 are disposed. The control blocks 21 extend substantially vertically from the LCD attachment plate 2 as much as a predetermined height, whereby the LCD attachment plate 2 and the handle mechanism section 4 are connected and fixed so that to sandwich the frame 3 therebetween. Next, the control blocks 21 are constituted of portions which extend from the substantially vertically extending portions to the oblique downside of the drawing (i.e., the lower end of the LCD) along a direction substantially horizontal to the LCD attachment plate 2. The portions of the control blocks 21 which vertically extend from the LCD attachment plate 2 are provided with attachment holes for fixing and connecting the portions to a bridge connecting portion 41 of the handle mechanism section 4 by fixing means such as screws. The portions of the control blocks 21 which substantially extend horizontally with respect to the LCD attachment plate 2 are used to engage with engagement portions 5121 each constituted of a cutout portion 516 of a first rotary block 51 and a cutout portion 523 of a second rotary block 52 described later. When the LCD is positioned at the lowermost end (an inspection operating position), the engagement portions 5121 are engaged with the control blocks 21, whereby relative rotation between the first rotary blocks 51 and the second rotary blocks 52 described later is stopped, and in consequence, integral rotation is possible.

Moreover, under each of the pair of control blocks 21, a stopper 22 having an L-shaped section is disposed. This stopper 22 is provided with a rising portion which is one side of the L-shape and engages with a stop groove 526 described later, whereby the LCD positioned at the lowermost end and during a rising movement is prevented from rotating downwardly to a horizontal position. The other side of the L-shape is fixed to the LCD attachment plate 2 by fixing means such as screws or an adhesive. The LCD attachment plate is provided with guide rollers 24 each having a columnar shape including a stepped portion where a dented intermediate stage is formed and having a screw on the upper surface thereof (in the present invention, four guide rollers on the upper, lower, right, and left sides are disposed, and the two guide rollers on the right and left sides rollers disappear behind the bridge connecting portion 41). The screws are screwed (or removed) to attach the guide rollers 24 to guide grooves 35 disposed in the frame 3. The guide rollers 24 and the guide grooves 35 constitute a guide mechanism. Needless to say, the mechanism is not limited to such a configuration, and a mechanism such as a slide rail of a conventional technology may be used.

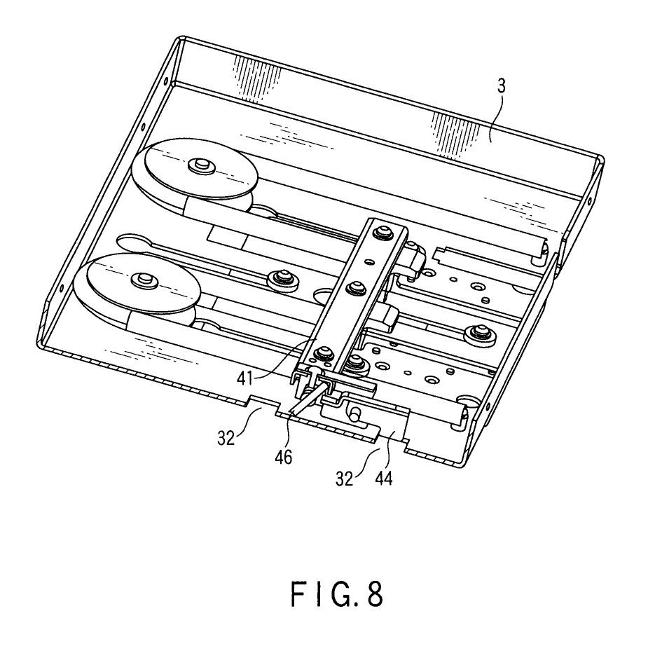

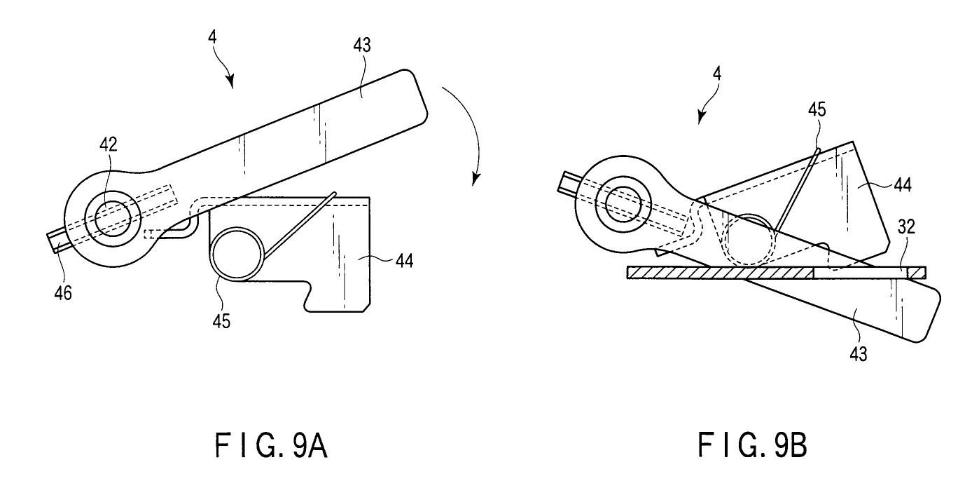

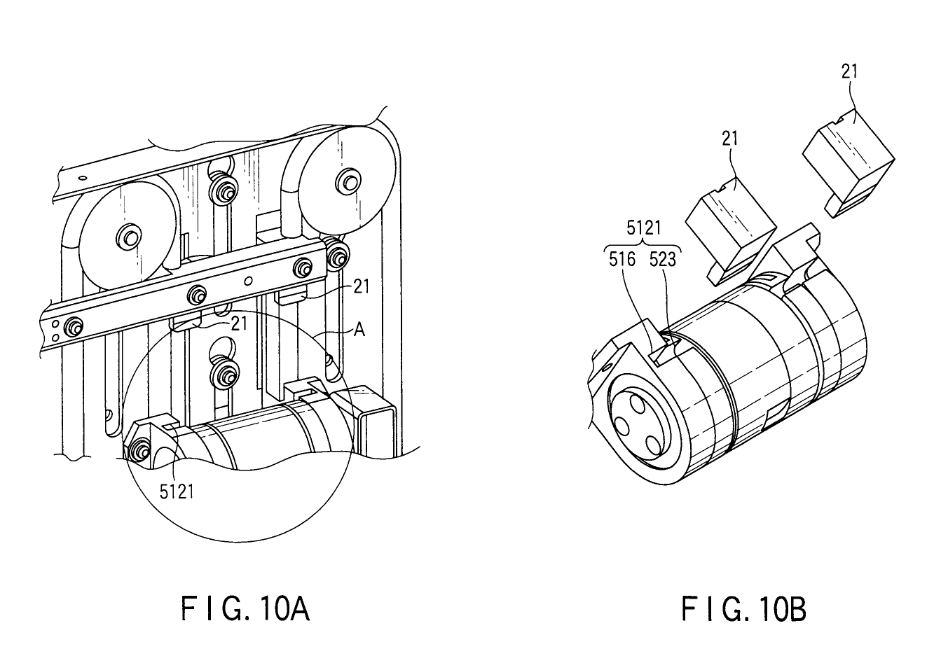

Moreover, as shown in FIG. 3, a grip portion 43 is connected to the control blocks of the LCD attachment plate 2 by the bridge connecting portion 41, whereby the handle mechanism section 4 can be guided together with the LCD 100 to move upwardly or downwardly with respect to the frame 3. As shown in FIGS. 8 and 9, the bridge connecting portion 41 is provided with a connection rod 42, a lock lever 44, a torsional spring 45, and a pressing rod 46 so that they can rotate. The torsional spring 45 is attached to the lock lever 44 and is always urged with respect to the lock lever 44 in such a direction as to engage with a lock hole 32 in the frame 3 described later. The pressing rod 46 is fixed and connected to the connection rod 42 together with the grip portion 43 so as to integrally rotate. The lock lever 44 has one end pressed by the pressing rod 46, and can rotate in such a direction as to be away from the lock hole 32 against the urging force of the torsional spring 45. The lock lever 44 and the lock hole 32 constitute a lock/unlock mechanism.

The LCD attachment plate 2, the frame 3, the lock/unlock mechanism, and the guide mechanism constitute a position selecting mechanism.

The handle mechanism section 4 operates as follows. When the grip portion 43 is pressed with a hand (in a direction shown by a blank arrow in FIG. 9A), the lock lever 44 has the one end pressed by the pressing rod 46 interlocked with the rotation of the grip portion 43 via the link 42, whereby the lock lever rotates in such a direction as to be away from the lock hole 32 against the urging force of the torsional spring 45 (FIG. 9B), i.e., in such a direction as to unlock the locked lever. When the grip portion 43 is released from the hand, the lock lever 44 is urged by the torsional spring 45 in such a direction as to fit into the lock hole 32, and finally fitted into the lock hole 32 to lock the lock lever 44. Needless to say, the present invention is not limited to the fitting lock mechanism, and any mechanism for fitting lock in a conventional technology may be used in the present invention.

Next, the frame 3 according to the present invention will be described with reference to the perspective view of FIG. 4. The frame 3 is formed into a substantially rectangular shape, four sides of the rectangular shape substantially vertically rise, and one of both the rising right and left sides (the left side in the drawing) is provided with a long hole 31 through which the connection rod 42 of the handle mechanism section 4 can pass, so that the connection rod 42 can slide along the long hole 31 in an upward-downward direction of the drawing. In a corresponding position of the frame 3 engaged with the lock lever 44 to lock the same, two upper and lower lock holes 32 are disposed. When the LCD is moved to the uppermost end (folding) position, the lock lever 44 fits into the upper lock hole 32 to lock the lever, and when the LCD is moved to the lowermost end (inspection operating) position, the lock lever 44 fits into the lower lock hole 32 to lock the lever.

Moreover, the frame 3 is provided with the guide grooves 35 corresponding to the guide rollers 24, and along the guide grooves 35, the guide rollers 24 are guided in the upward-downward direction. Thus, the guide rollers 24 and the guide grooves 35 constitute a guide section. There is not any special restriction on the positions and the number of the guide grooves, as long as the guide grooves correspond to the guide rollers. Needless to say, the frame 3 is provided with block through holes 36 through which the control blocks 21 and the stopper 22 pass to fix the bridge connecting portion 41 to the control blocks 21, and the positions and the number of the block through holes 36 also correspond to those of the control blocks and the stopper, and the control blocks 21 and the stopper 22 may upwardly or downwardly slide along the block through holes so that the LCD can slide to the uppermost end or the lowermost end.

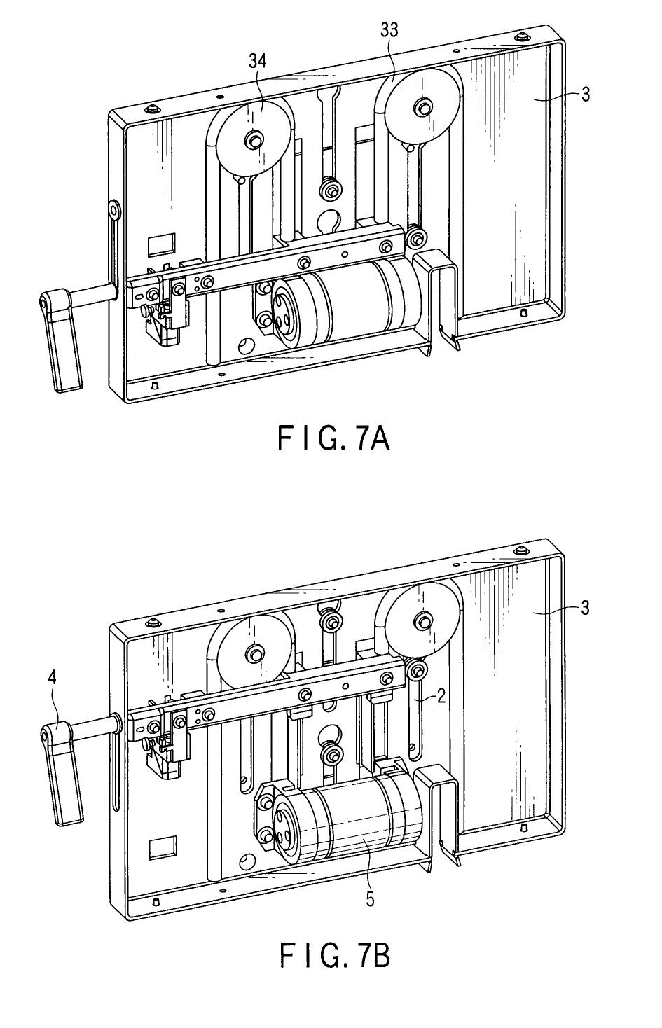

As shown in FIGS. 7A and 7B, each of a pair of tensile springs 33, via each of a pair of pulleys 34, has one end fixed and connected to a position near to the bottom end of the frame 3, and has the other end connected to the control block 21 disposed substantially in the center of the LCD attachment plate 2, to offset the weight of the LCD, thereby realizing the uniformizing of an operation force for upwardly or downwardly moving the LCD and the stabilization of an operation of the lock lever 44 at the upper/lower position. Moreover, the tensile springs 33 are formed in a U-shape by the pulleys 34, whereby a necessary spring length is assured, and a spring constant is decreased, so that the operation force is further uniformized.

Hereinafter, the rotation mechanism section 5 which rotatably supports the LCD 100 and the LCD attachment plate 2 and the frame 3 of the support mechanism main body 1a will be described in detail. FIG. 5 is an exploded perspective view of the rotation mechanism section 5. FIG. 6 is a transverse sectional view of the rotation mechanism section 5. As shown in FIGS. 5 and 6, the rotation mechanism section 5 mainly comprises a pair of first rotary blocks 51, a pair of second rotary blocks 52, and a third rotary block 53. The pair of second rotary blocks 52 are disposed symmetrically with respect to the third rotary block 53, and the pair of first rotary blocks 51 are also disposed symmetrically with respect to the third rotary block 53 so as to sandwich the second rotary blocks 52 therebetween. The third rotary block also has a right-left symmetric structure. Moreover, structures disposed in the first to third rotary blocks which will be described later are symmetrically present, but it is unnecessary to further emphasize that each of the structures forms “a pair”.

As shown in FIGS. 5 and 6, the first rotary block 51 is integrally constituted of a semicircular portion 510 and a rectangular portion 512. On the side surface of the rectangular portion 512, a planar attachment portion 513 to be attached to the frame 3 is disposed, and the planar attachment portion 513 is provided with attachment holes for fixing and attaching the first rotary block 51 to the frame 3 by fixing means such as screws. The first rotary block 51 is provided with a through hole 514 along an axial direction of the rotation mechanism section 5, through which a plug 50 and a shaft 534 of the third rotary block 53 described later are inserted. In the end face of the first rotary block 51 on the side of the semicircular portion 510 along the same axial direction as that of the through hole 514 outside the through hole in a diametric direction, a circular second guide groove 511 for guiding a second pin 522 of the second rotary block 52 described later is disposed. Moreover, the upside of the first rotary block 51 in the drawing is provided with the cutout portion 516 which constitutes the engagement portion 5121 together with the cutout portion 523 of the second rotary block 52 as described later so as to engage with the control block 21 of the LCD attachment plate 2, thereby integrally rotating the first rotary block 51 and the second rotary block 52.

As shown in FIGS. 5 and 6, the second rotary block 52 has a substantially circular shape. One end face of the second rotary block facing the first rotary block 51 in the axial direction is provided with the second pin 522 to be inserted into the second guide groove 511, whereas the other end face thereof is provided with a first pin 521 to be inserted into a first guide groove 531 of the third rotary block 53 described later, and a torsional spring fixing hole 525 into which the end of a torsional spring 533 is inserted as described later. Needless to say, in the axial center of the second rotary block 52, a through hole 524 is disposed into which the shaft 534 is fitted via a bearing or the like as described later. In the embodiment, as shown in FIG. 6, the shaft 534 passes through the through holes 514 and 524 to rotatably fit into a shaft of the plug 50 fixed to the other end face of the first rotary block 51 in the axial direction.

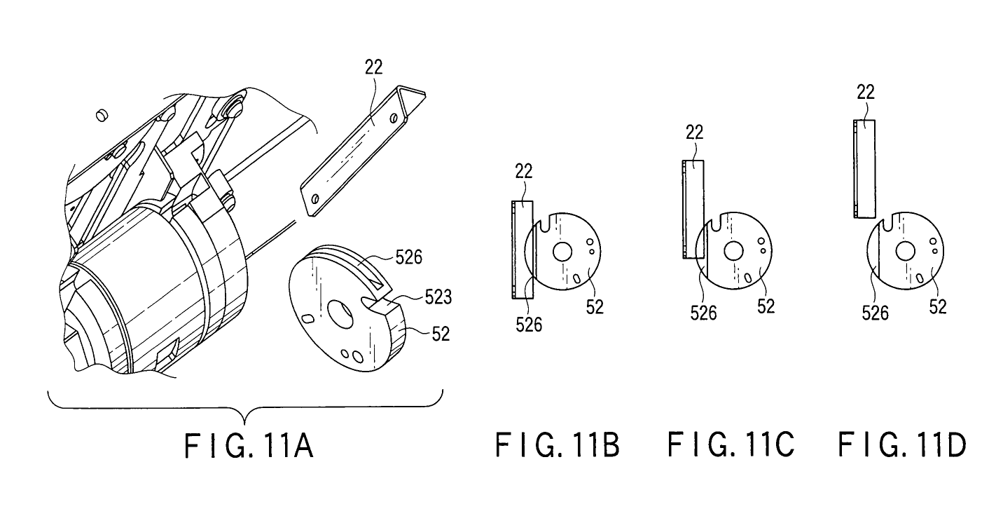

Moreover, the stop groove 526 may be disposed along the outer peripheral surface of the second rotary block 52 in the upward-downward direction, and the stop groove 526 has a U-shaped section in a radial direction (the sectional shape is not limited to this shape, and may be another shape as long as the stopper can fit into the groove). When the LCD is positioned at the lowermost end and in a rising process of moving upwardly from the lowermost end, the stopper 22 of the LCD attachment plate 2 fits into the stop groove 526 to limit the rotary angle of the LCD, thereby preventing the LCD from being rotated to the horizontal position at this time. Moreover, only when the LCD moves to the uppermost end and the stopper 22 is completely disposed away from the stop groove, the LCD rotates to the horizontal position. The operation is specifically shown in FIGS. 11A to 11D. FIGS. 11A to 11D are operation explanatory views showing mutual movement and fitting relation between the stopper 22 and the stop groove 526. The stopper and the stop groove have a fitted state shown in FIG. 11B when the LCD is positioned at the lowermost end, have a fitted state shown in FIG. 11C in the rising process, and have a fitted state shown in FIG. 11D when the LCD is positioned at the uppermost end. It is seen from the drawings that in the states FIGS. 11B and 11C, the second rotary block 52 engages with the stopper and cannot rotate with respect to the frame 3.

Moreover, the control blocks 21 and the stopper 22 attached to the LCD attachment plate 2, the engagement portions 5121 in the first rotary blocks 51 and the second rotary blocks 52, and the stop grooves 526 of the second rotary blocks 52 constitute a rotation block mechanism.

As shown in FIGS. 5 and 6, the third rotary block 53 has a substantially columnar shape, and a pair of ring-like torsional spring fixing grooves 536 for fixing the torsional springs 533 are symmetrically disposed in the axial direction. In the torsional spring fixing grooves 536, the torsional springs 533 are fixed. Moreover, when one end of each torsional spring projects from the end face of this groove and is inserted into the torsional spring fixing hole 525, the torsional spring 533 offsets a moment generated by the own weight of the LCD. In the center of the ring-like torsional spring fixing groove 536, the shaft 534 of the third rotary block 53 is disposed, and the shaft 534 extends along the shaft of the third rotary block 53 to the through hole 514 of the first rotary block 51. This shaft is inserted through a disc spring 532, and the disc spring 532 is positioned between the second rotary block and the third rotary block in the axial direction. A pressure in the axial direction is generated between the blocks to increase a frictional force therebetween, thereby preventing the problem of the rotation of the rotation mechanism owing to the own weight of the LCD, and controlling an operation force during the rotation. Needless to say, the present invention is not limited to the disc spring 532, and another member may be used as long as the frictional force between the second rotary block and the third rotary block can be increased.

Moreover, the first guide groove 531 to be engaged with the first pin 521 is disposed outside the torsional spring fixing groove of the third rotary block 53 in the radial direction of the third rotary block 53. Moreover, the third rotary block 53 is fixed or integrally connected to the support arm 1b rotatably supported on the apparatus main body 400.

Therefore, as shown in FIGS. 7A and 7B, the LCD 100 fixed to the LCD attachment plate 2 is slidably attached to the frame 3 of the support mechanism main body 1a, and the handle mechanism section 4 is fixed and connected to the LCD attachment plate so that the section can upwardly or downwardly slide together with the plate along the frame 3. The rotation mechanism section 5 is fixed to the frame 3 via the planar attachment portion 513, whereby the LCD 100, the LCD attachment plate 2, the frame 3, and the handle mechanism section 4 can entirely rotate in the upward-downward direction with respect to the support arm 1b.

Hereinafter, an operation of the support mechanism 1 of the embodiment will be described with reference to FIGS. 7, 12, and 13.

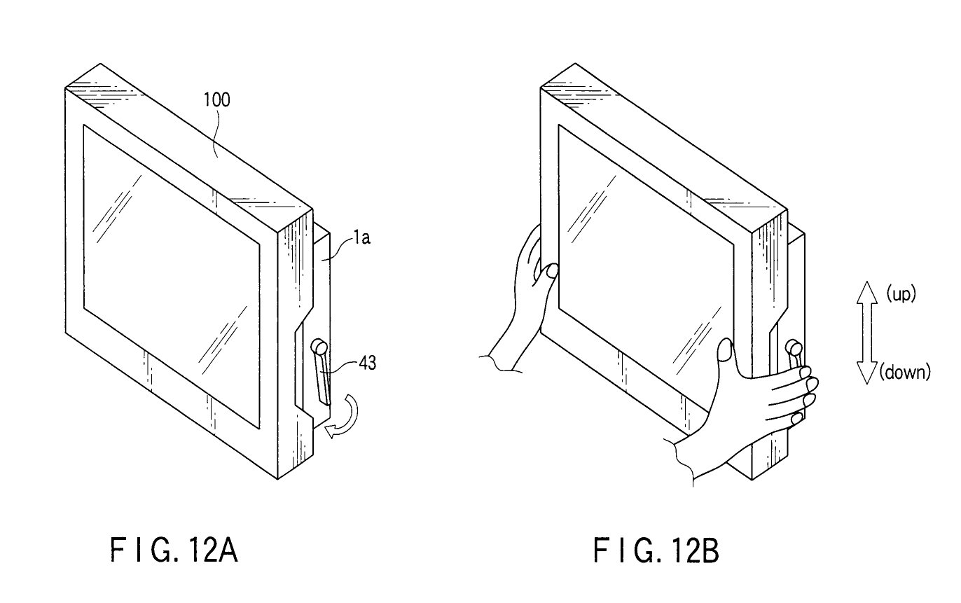

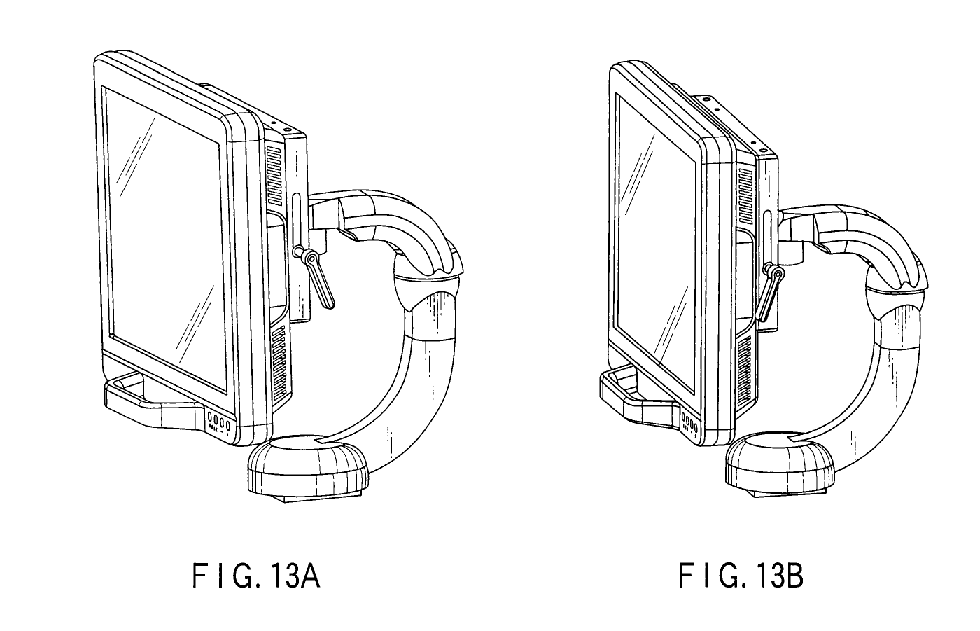

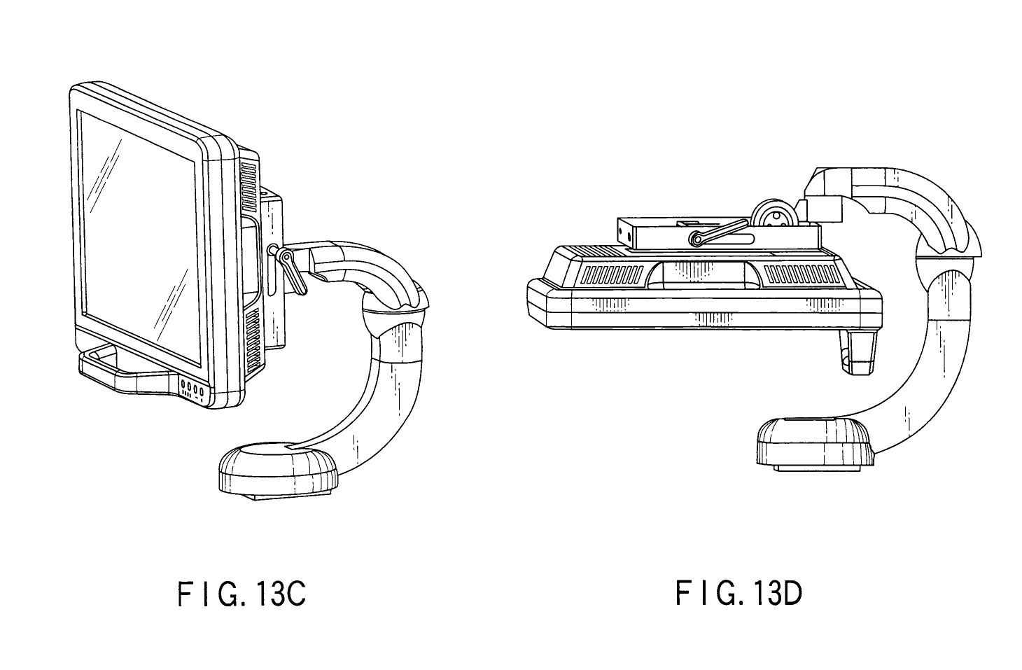

First, as shown in FIGS. 7A and 13A, the LCD is positioned at the lowermost observing position of the LCD display surface, i.e., at the lowermost end, and the lock lever 44 of the handle mechanism section 4 engages with the lower lock hole 32 formed in the frame 3 to lock by the torsional spring 45. At this time, the handle mechanism section 4 is locked and cannot move, and hence the LCD attachment plate 2 and the LCD 100 fixed and connected to the handle mechanism section 4 are also locked and cannot move upwardly or downwardly. At this time, when the LCD is rotated upwardly or downwardly, the control blocks 21 of the LCD attachment plate 2 engage with the engagement portions 5121 each constituted of the cutout portion 516 of the first rotary block and the cutout portion 523 of the second rotary block 52 of the rotation mechanism section 5, whereby the first rotary blocks 51 and the second rotary blocks 52 integrally rotate. Moreover, as shown in FIG. 5, the first rotary blocks 51 and the second rotary blocks 52 integrally rotate with respect to the third rotary block 53. At this time, the second rotary blocks 52 rotate with respect to the third rotary block 53, and the first rotary blocks 51 and the second rotary blocks 52 relatively stand still. Therefore, the fitting of the first pin 521 into the first guide groove 531 works, whereas the fitting of the second pin 522 into the second guide groove 511 does not work. At this time, when the peripheral length of the first guide groove 531 is set to such an extent that, for example, the second rotary block 52 is rotated upwardly by 25° and downwardly by 10°, the rotary range of the second rotary block can be limited to 35°. Needless to say, the rotary range is limited to a desirable angular range by changing the peripheral length of the first guide groove 531.

Next, an operator rotates the LCD with a force in a direction shown by a blank arrow in FIG. 12A to push the lock lever 44 from the lock hole 32 against the urging force of the torsional spring 45, thereby unlocking the locked handle mechanism section 4, so that the handle mechanism section can move upwardly or downwardly. At this time, when the operator manually presses the grip portion 43 of the handle mechanism section 4 in the arrow direction while raising the LCD, the LCD 100, the LCD attachment plate 2, and the handle mechanism section 4 integrally move upwardly with respect to the frame 3. In this way, the LCD and the like fixed to the handle mechanism section 4 move upwardly by a manual impulsive force (see FIG. 12B). At this time, the engagement of the control block 21 and the engagement portion 5121 is unlocked. However, as shown in FIG. 11C, the stopper 22 still fits into the stop groove 526 so that the stopper slides with respect to the stop groove until the LCD reaches the uppermost end position. Since the second rotary blocks 52 still cannot rotate together with the first rotary blocks 51 with respect to the frame 3, as described above, the only first pin 521 operates, and the first pin 521 fitted into the first guide groove 531 limits the rotary angle of the LCD 100. Therefore, even if the engagement of the control block 21 and the engagement portion 5121 is unlocked at this time, the LCD 100 does not rotate by 90° before reaching the uppermost end position, which acquires the stability of the rising operation of the LCD.

Finally, when the LCD 100 rises to the highest position (FIG. 7B), the engagement of the stopper 22 and the stop groove 526 is unlocked (see FIG. 11D). Therefore, the frictional force is hardly exerted between the first rotary block 51 and the second rotary block 52, and the disc spring 532 for increasing the frictional force is disposed between the second rotary block and the third rotary block, whereby the second rotary block 52 rotates together with the third rotary block 53 with respect to the first rotary block fixed to the frame 3. At this time, the second pin 522 mainly operates, and fits into the second guide groove 511 disposed in the first rotary block 51, to limit the rotary angle of the LCD. In this case, when the peripheral length of the second guide groove 511 is set to such an extent that the first rotary block 51 rotates upwardly by 25° and downwardly by 90° with respect to the second and third rotary blocks, the rotary range of the first rotary block can be limited to 115°. In consequence, the LCD 100 rotates as shown in FIG. 13D. Needless to say, the rotary range is limited to a desirable angular range by changing the peripheral length of the second guide groove 511. The above guide grooves and the pins for the guides constitute a rotary range limiting section.

In this way, a switching operation from the inspection operating state to the folded state of the LCD is realized. Needless to say, an operation from this folded state to the inspection operating state is only reverse to the above operation, and hence the operation is not described in further detail.

According to the first embodiment, during the inspecting operation, the operator sets the image display device to the lowermost end position suitable for the inspecting operation, whereby the image display device can be rotated in a small rotary angle range and adapted to an observation angle. In a case where the apparatus needs to be conveyed and hence the image display device is folded, the image display device is merely moved upwardly to change the rotary angle range, whereby the device can be rotated downwardly by 90° and folded. Moreover, when the image display device is moved upwardly to the uppermost end position, the device is not rotated downwardly by 90°. Therefore, the operability of the regulation of the image display surface during diagnosis/inspection is not influenced, but the image display surface of the image display device during movement/storage can be reclined downwardly, whereby device movability can be improved to prevent dirt or damage on the image display surface. Moreover, a burden onto the operator is not increased, but comfort during the observation can be given to the operator.

The above lowermost end position is a position of the image display device 100 at the lowermost end in a movement region where the device rises or lowers with respect to the rotation mechanism section 5, and in this case, the guide rollers 24 are positioned at the lowermost ends of the guide grooves 35. The above uppermost end position is a position of the image display device 100 at the uppermost end in the movement region where the device rises or lowers with respect to the rotation mechanism section 5, and in this case, the guide rollers 24 are positioned at the uppermost ends of the guide grooves 35.

However, in a process of lowering from the uppermost end position to the lowermost end position, the image display device 100 rapidly lowers owing to its own weight, and the guide rollers 24 collide with the bottom ends of the guide grooves 35, thereby easily causing a problem such as break-down of a display screen which an easily crushing member. Therefore, the operator needs to carefully, slowly, and persistently apply a force smaller than the upward raising force with respect to the image display device 100, which brigs discomfort during the operation. Moreover, an operator who first operates the device does not apply a uniform force, which easily cause a damage on the image display device 100 or the like.

The present inventors have conducted further researches in view of the above problems, and have eventually suggested a lowering speed suppression mechanism K installed in the ultrasonic diagnosis apparatus, in which the lowering speed of the lowering image display device 100 is suppressed to lower the lowering speed as compared with a free speed at which the image display device 100 lowers owing to its own weight.

Hereinafter, the lowering speed suppression mechanism K will be described with respect to examples.

### Example 1

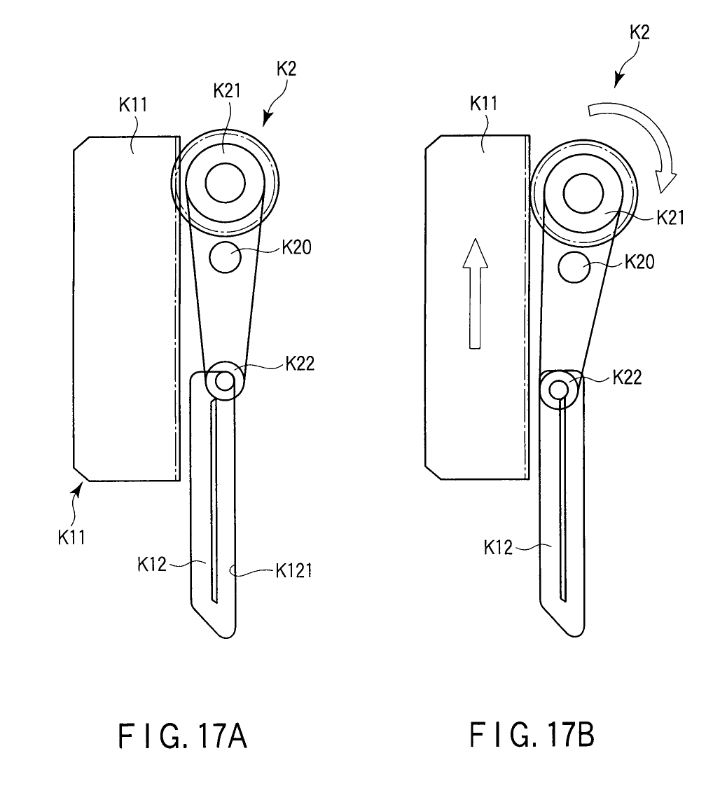

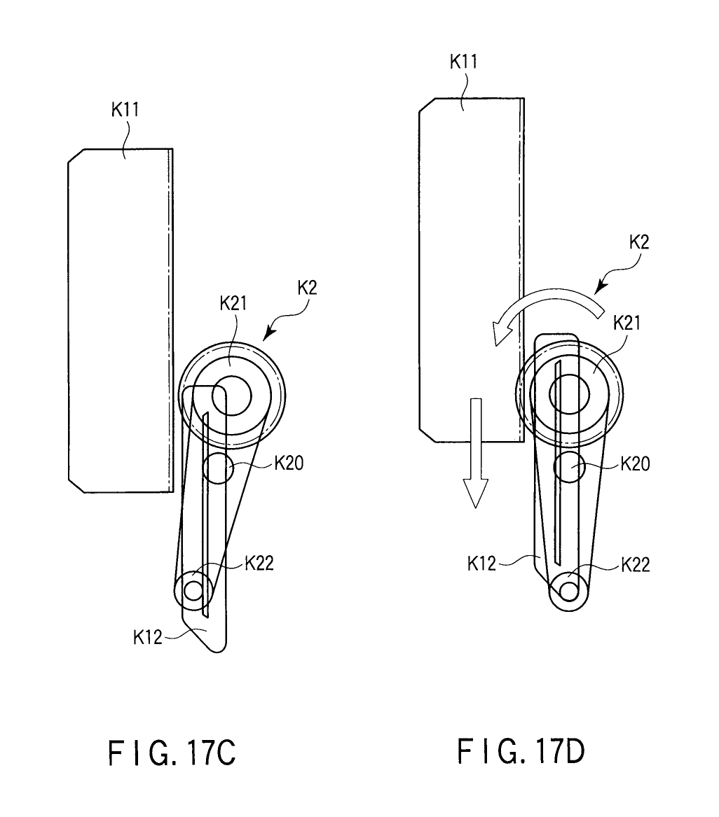

Example 1 of a lowering speed suppression mechanism K will be described with reference to FIG. 17. FIG. 17 is a so-called side view which can entirely explain relative movement of members to clearly show a structure of the lowering speed suppression mechanism K. Here, another drawing is omitted.

To an LCD attachment plate 2, there are fixed a rack K11 extending in a rising/lowering direction of an image display device 100, and a cam K12. The rack K11 comprises teeth formed on the side of the rack K11 facing a pinion K21 described later, and the cam K12 is formed outside the lower end of the rack K11 so as to come close to the teeth of the rack K11. Moreover, the cam K12 comprises a guide groove K121, and the guide groove K121 has a closed ring-like shape, and guides a pulley K22 described later. The rack K11 and the cam K12 constitute an image display device side fixing member K1.

A lever K2 which is a support mechanism side urging member is fixed to a frame 3. Specifically, the lever K2 is rotatably fixed to the frame 3 via a supporting point K20 positioned substantially in the center of the lever. The lever K2 comprises one end having the pinion K21 which can rotate around the axial center thereof with the decay of the rotational velocity to engage with the teeth of the rack K11, and the other end having the pulley K22 fitted into the guide groove K121 so that the pulley is guided.

As shown in FIG. 17A, when an inspecting operation is performed by using the image display device 100, the image display device 100 is disposed at the lowermost end position, the upper-end teeth of the rack K11 engage with the pinion K21, and the pulley K22 is positioned in the upper right corner of the guide groove K121 of the cam K12.

Next, as shown in FIG. 17B, when the image display device 100 starts rising, the rack K11 applies, to the pinion K21, a force for rotating the pinion clockwise. Since the pinion K21 decays around the axial center, the pinion does not easily rotate, and hence the pinion K21 applies a rightward force to one end of the lever K2 in which the pinion is included. In consequence, the pulley K22 at the other end of the lever slides along the guide groove K121 toward the left to horizontally slide to the upper left corner of the groove. In consequence, the pinion K21 is away from the teeth of the rack K11 and is positioned outside the teeth, and the pinion does not apply any force to the rack K11, whereby the image display device 100 can favorably rise.

As shown in FIG. 17C, when the image display device 100 rises, the pulley K22 linearly slides along the guide groove K121 downwardly from the upper left corner. Immediately before the image display device 100 reaches the uppermost end position, the pulley K22 is positioned in the lower left corner of the cam K12, and the pinion K21 is positioned outside the lower end of the rack K11. When the image display device 100 continuously rises, the pulley K22 linearly slides to the lower right corner obliquely under the lower left corner. When reaching the lower right corner, the pinion K21 completely engages with the rack K11, whereby the image display device 100 reaches the uppermost end position (i.e., the uppermost end position in the movement region where the image display device rises or lowers).

Next, as shown in FIG. 17D, when the image display device 100 lowers from the uppermost end position, the movement of the pulley K22 is limited in the lower right corner of the guide groove K121, and the pulley cannot move to the right or the downside, whereby the pinion K21 is constantly engaged with the telephoto end of the rack K11. Since the pinion K21 decays around the axial center, an engaging speed of the rack K11 and the pinion K21 is low. The lowering speed of the image display device 100 is determined by the engaging, and the lowering speed of the image display device 100 is suppressed to be lower than that of the device owing to its own weight.

Moreover, when the image display device 100 lowers, the cam K12 fixed to the LCD attachment plate 2 also lowers together with the image display device 100, and hence the pulley K22 linearly slides upwardly with respect to the guide groove K121. When the image display device 100 finally lowers to the lowermost end position, the rack K11, the cam K12, and the pinion K21 return to the state shown in FIG. 17A.

As described above, the guide groove K121 of the cam K12 is formed in a quadrangular shape having four corners, so that the pulley K22 is guided to perform an operation along the above locus. Moreover, to ensure that the engaging of the rack K11 and the pinion K21 and cooperation of the cam K12 and the pulley K22 do not involve mutual interference, the rack K11 and the cam K12 are disposed on mutually deviated parallel planes, the pinion K21 is disposed on the plane where the rack K11 exists, the pulley K22 is disposed on the plane where the cam K12 exists, and the lever K2 is preferably disposed between the two planes.

### Example 2

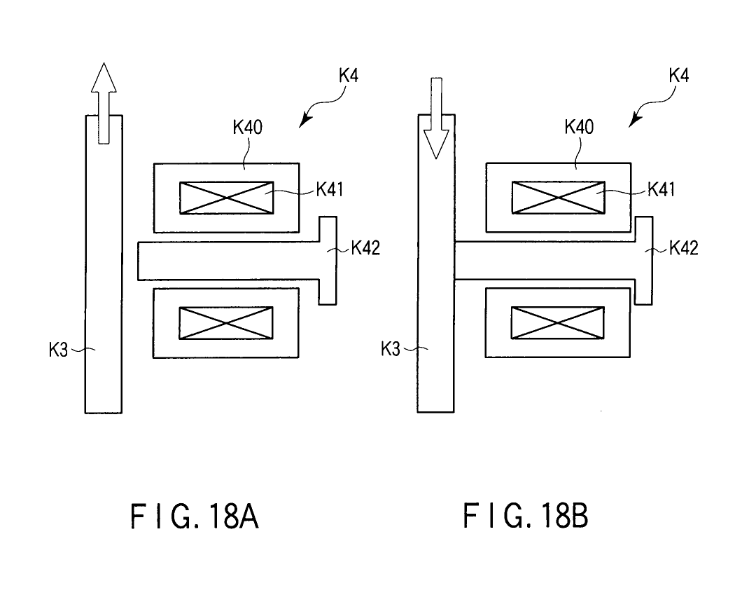

Example 2 of a lowering speed suppression mechanism K will be described with reference to FIGS. 18A and 18B.

A rail K3 extending in a rising/lowering direction of an image display device is fixed to an LCD attachment plate 2, and the only rail K3 is an image display device side fixing member. A side surface of the rail K3 adjacent to the surface thereof fixed to the LCD attachment plate 2 is a rail surface. The rail surface is preferably a rough surface.

A support mechanism side urging member fixed to a frame 3 is constituted of an electromagnetic coil K41 which is a rotor, a post K42 which is a movable piece, and a coil frame K40 in which the electromagnetic coil K41 is disposed. When the electromagnetic coil K41 fixed to the frame 3 by use of the coil frame K40 as a supporting point is excited, the post K42 comes in contact with the rail surface of the rail K3. When the excitation of the electromagnetic coil K41 is canceled, the post K42 is away from the rail surface.

As shown in FIG. 18A, when an image display device 100 starts rising from the lowermost end position, the excitation of the electromagnetic coil K41 is canceled, the post K42 is away from the rail K3, and any force is not exerted to the rail K3, but the image display device 100 favorably rises.

As shown in FIG. 18B, when the image display device 100 starts lowering from the uppermost end position, the electromagnetic coil K41 is excited, and the post K42 comes in contact with the rail surface of the rail K3 to apply an upward frictional force to the rail K3, thereby lowering the lowering speed of the rail K3. The lowering speed of the image display device 100 is determined by that of the rail K3, and hence the lowering speed of the image display device 100 is suppressed to be lower than the lowering speed of the device owing to its own weight.

### Example 3

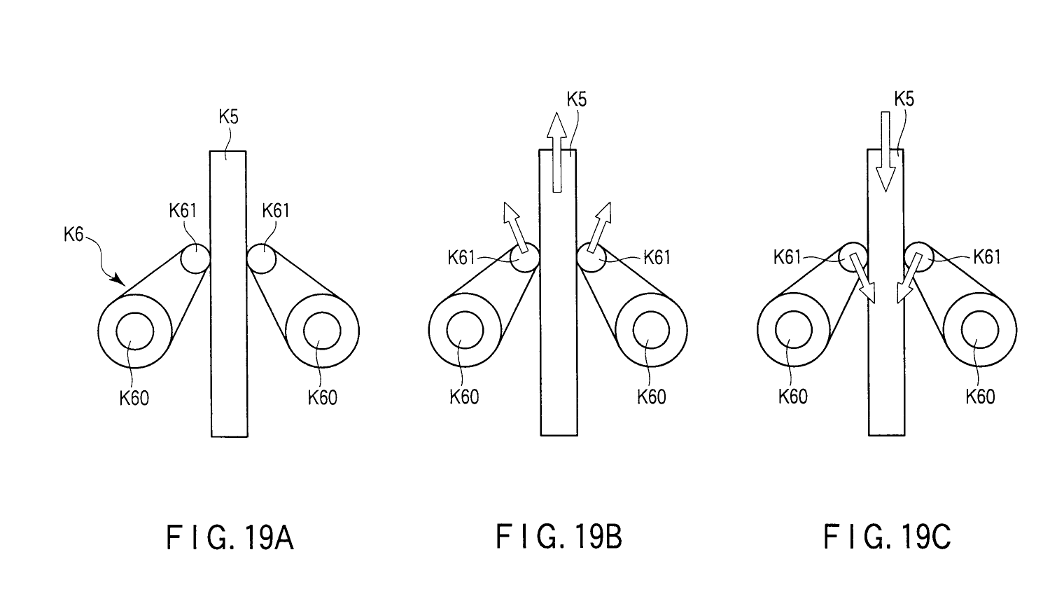

Example 3 of a lowering speed suppression mechanism K will be described with reference to FIGS. 19A, 19B, and 19C.

A rail K5 extending in a rising/lowering direction of an image display device is fixed to an LCD attachment plate 2, and the only rail K5 is an image display device side fixing member. Two side surfaces adjacent to the surface of the rail K5 fixed to the LCD attachment plate 2 are rail surfaces.

Two levers K6 and K6 which are support mechanism side urging members are fixed to a frame 3. Specifically, the levers K6 and K6 are rotatably fixed to the frame 3 via supporting points K60 and K60 each of which is one end of each lever. Other inwardly facing ends K61 and K61 of the levers K6 and K6 come in contact with the two rail surfaces of the rail K5. The ends which become the supporting points K60 and K60 are farther from the rail surfaces than the ends K61 and K61. Therefore, the two levers K6 and K6 form a roof-like shape which makes it possible to sandwich the rail K5 between the levers. Moreover, the ends K61 and K61 can elastically be deformed.

As shown in FIG. 19A, when an image display device 100 is positioned at the lowermost end position, the ends K61 and K61 do not move with respect to the rail K5, and the ends K61 and K61 do not cause elastic deformation.

As shown in FIG. 19B, when the image display device 100 rises, the rail K5 applies an upward force to the other ends K61 and K61. The two levers K6 and K6 rotate away from the rail surfaces in a counterclockwise direction around the supporting points K60 and K60. However, the center of gravity of each of the levers K6 and K6 is disposed between the supporting point K60 and the end K61, whereby the levers K6 and K6 rotate clockwise around the supporting points K60 and K60 owing to a force of gravity, and the ends K61 and K61 just moved away form the rail surfaces come close to the rail to abut on the rail surfaces again. In this way, in the rising process of the image display device 100, the two levers K6 and K6 repeatedly come in contact with and move away from the rail surfaces, and an urging force to the rail K5 is decreased, whereby the image display device 100 can comparatively favorably rise.

As shown in FIG. 19C, when the image display device 100 rises to the uppermost end position and starts lowering, the rail K5 applies a downward force to the ends K61 and K61. The two levers K6 and K6 rotate clockwise around the supporting points K60 and K60, respectively, so as to come close to the rail K5, and the levers press the rail surfaces to cause the elastic deformation. A resisting force is applied to the rail K5 by the elastic deformation, to lower a lowering speed of the rail K5. The lowering speed of the LCD 100 is determined by that of the rail K5, and hence the lowering speed of the image display device 100 can be suppressed to be lower than that of the device owing to its own weight.

According to the above examples, the lowering speed suppression mechanism K can be disposed to suppress the lowering speed of the image display device 100 during the lowering, whereby break-down of the image display device or the like can be prevented.

### Second Embodiment

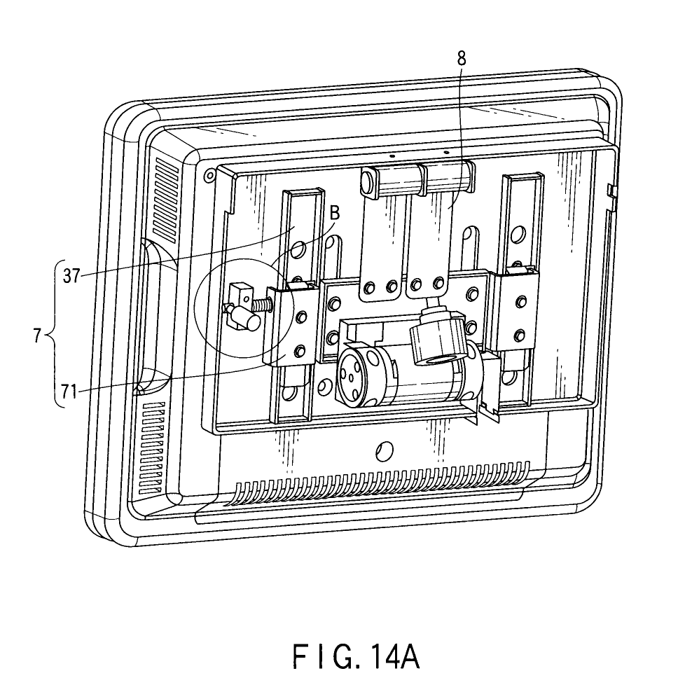

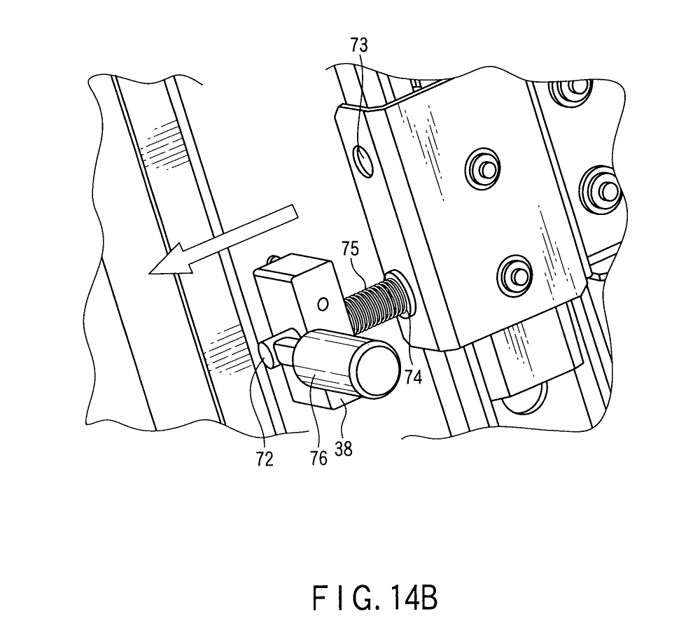

Hereinafter, a second embodiment will be described with reference to FIGS. 14A and 14B. In the present embodiment, detailed drawing and description of the same part as the above first embodiment is omitted, and here an only different part will be described.

In the second embodiment, a pair of Conston springs 8 are used in place of the tensile springs 33 and the pulleys 34 of the first embodiment, and a rail mechanism 7 is disposed in place of the handle mechanism section 4. Reels of the Conston springs 8 are substantially fixed to the center of the upper end of the frame 3, and drawn free ends are fixed to a guide plate 71 of the rail mechanism 7. The rail mechanism 7 comprises the guide plate 71 and a pair of rails 37 fixed to the frame 3. The guide plate 71 is fixed and connected to control blocks 21 of an LCD attachment plate 2 by fixing means such as screws, and is provided with guide portions extending in an upward-downward direction on right and left sides in the drawing, whereby the rails 37 guides the guide plate 71 so as to extend through the guide portions. The guide plate 71 and the rails 37 constitute another guide mechanism.

Moreover, in one of the side surfaces of the guide plate 71 vertical to the frame 3 at right and left ends (the left side surface in the present invention), two positioning holes 73 and 74 are disposed, and the two positioning holes 73 and 74 correspond to the uppermost end position and the lowermost end position of an LCD, respectively. The frame 3 is further provided with a fixing block 38, and the fixing block 38 is provided with a through hole in a direction of the above positioning holes, i.e., in a right-left direction of the drawing. One side of an L-shaped traction rod 72 passes through this through hole corresponding to the uppermost end position or the lowermost end position of the LCD, and fits into each of the positioning holes 73 and 74. A shaft of the L-shaped traction rod 72 which becomes the one side thereof is provided with a tensile spring 75 between the fixing block 38 and the side surface of the guide plate 71 provided with the positioning holes, and the tensile spring 75 constantly urges the traction rod 72 in a direction in which one end of the traction rod 72 is inserted into the positioning hole. The other side of the L-shaped traction rod 72 is provided with a traction button 76, whereby an operator grips the traction button 76 to pull the traction rod 72 in a direction shown by a blank arrow in FIG. 14B. The traction rod 72 and the positioning holes 73 and 74 constitute another lock/unlock mechanism.

The rail mechanism 7 of the second embodiment operates as follows. That is, when an LCD 100 is positioned at the lowermost end and the traction button 76 is pulled in the direction shown by the arrow, one end of the traction rod 72 is pulled out of the lower positioning hole 74 against the urging force of the tensile spring 75. At this time, the traction button 76 can be pulled, and the LCD can be moved upwardly. When the LCD moves to the uppermost end position, the traction button 76 is loosened, and the traction rod 72 is inserted into the upper positioning hole 73 by the urging force of the tensile spring 75, thereby moving the LCD 100 to the uppermost end position. The next folding operation is the same as that in the first embodiment, and is not described here again.

According to the above structure, the present embodiment can produce the same function and effect as those of the first embodiment. Moreover, in the present embodiment, the Conston springs 8 are used in place of the tensile springs 33, whereby a more uniform operation force can be obtained.

### Third Embodiment

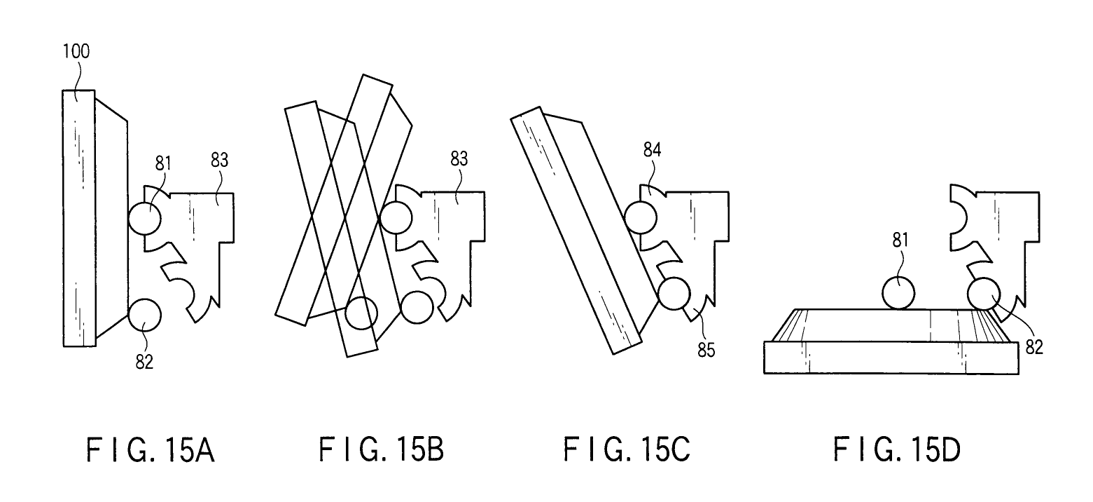

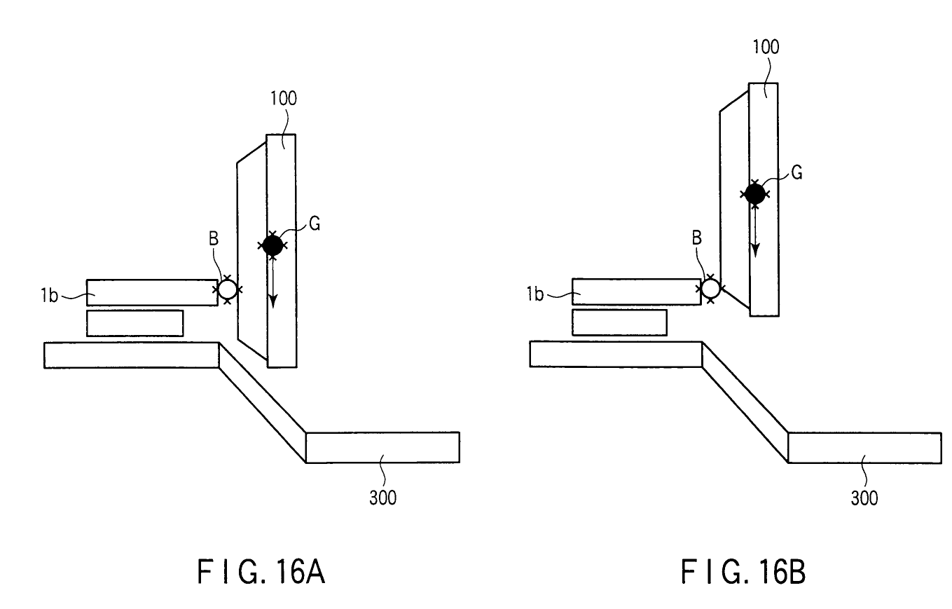

Hereinafter, a third embodiment will briefly be described with reference to FIGS. 15A, 15B, 15C, and 15D. In the present embodiment, detailed drawing and description of the same part as the above first embodiment is omitted, and here an only different part will be described.

In the third embodiment, a structure is installed as follows in place of the support mechanism main body 1a of the first embodiment. That is, two rotary shafts 81 and 82 arranged in an upward-downward direction are fixed to or formed integrally with an LCD attachment plate 2 (not shown). The upper rotary shaft 81 for use during inspection/diagnosis is disposed near the gravity center position of an LCD 100, and the lower rotary shaft 82 for use during movement/storage is disposed under the upper rotary shaft. The two rotary shafts can use the following structure. That is, a part corresponding to the first rotary block of the first embodiment is fixed to the LCD attachment plate 2, a part corresponding to the third rotary block of the first embodiment is fixed and fitted into shaft attachment washers 84 and 85 described later (shrink fit), and a part corresponding to the second rotary block of the first embodiment is omitted. Fitting of guide pins into guide grooves in the part corresponding to the first rotary block and the part corresponding to the second rotary block sets each of rotary ranges of the upper rotary shaft 81 and the lower rotary shaft 82 to a rotary range when the LCD is positioned at the uppermost end or the lowermost end in the first embodiment. A shaft connecting portion 83 is made of an elastic material such as a plastic material, has an attachable/detachable fitting mechanism in which the rotary shaft 81 or 82 can be attached or detached by a predetermined force, and comprises the two upper and lower shaft attachment washers 84 and 85 to which the upper and lower rotary shafts 81 and 82 are attached. The shaft attachment washers 84 and 85 have semi-columnar surfaces which coincide with semi-circular peripheral surfaces of the rotary shafts 81 and 82, and have a radius which is to be slightly smaller than that of each rotary shaft.

The structure of the third embodiment operates as follows. During inspection/diagnosis, the only upper rotary shaft 81 is inserted and fitted into the shaft connecting portion 83 (see FIG. 15A). During movement/storage, first the upper and lower rotary shafts 81 and 82 are fitted into the shaft attachment washers 84 and 85 (see FIGS. 14B and 14C), and then the fitting of the upper rotary shaft 81 into the shaft attachment washer 84 is unlocked (see FIG. 14D). In this way, the LCD can be rotated and reclined downwardly by using the lower rotary shaft 82 as a rotary shaft.

According to the above structure, the present embodiment can produce the same function and effect as those of the first embodiment. Moreover, in the present embodiment, the constitution of the support mechanism main body can further be simplified by using the fitting structure of the two rotary shafts 81 and 82 fixed to the LCD attachment plate 2 into the shaft connecting portion 83.

It is to be noted that the first to third embodiments may have modifications as follows. That is, in place of the structure where the first and second pins for guiding are disposed in the second rotary block 52 and the third rotary block 53 and the first rotary block 51 are provided with the first and second guide grooves, the first and second pins for guiding may be disposed in the third rotary block 53 and the first rotary block 51, respectively, and the second rotary block 52 may be provided with the first and second guide grooves corresponding to the first and second pins, respectively.

Needless to say, the present invention can be changed by a person with ordinary skill in another manner without requiring any creative labor. The above embodiments are merely examples for more easily understanding the present invention, and do not limit the present invention. Therefore, the components and parts disclosed in the above embodiments can be replaced with those that perform the same functions, designed anew or modified within the scope. Furthermore, any possible combination of these components or parts is included in the scope, as long as advantages are similar to those of the embodiments of the present invention.

According to the above description, various improvements and alterations of the present invention are possible. Therefore, it should be understood that even if the description specified herein is excluded, the present invention can be realized in another configuration within the accompanying claims.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnosis apparatus comprising: an image display device, an ultrasonic probe, an operation panel, an apparatus main body and a support mechanism,
wherein the support mechanism comprises a support mechanism main body and a support arm, the image display device is attached to the support mechanism main body, and supported on the apparatus main body by the support arm, and
the support mechanism main body is provided with:
a rotation mechanism which rotates the image display device in an upward-downward direction;
a position selecting mechanism which raises or lowers the image display device with respect to the rotation mechanism; and
a rotation block mechanism which blocks such rotation as to recline the image display device to a horizontal position in a case where the image display device is disposed at a position other than an uppermost end position in a movement region where the image display device rises or lowers with respect to the rotation mechanism.

2. The ultrasonic diagnosis apparatus according to claim 1, wherein the position selecting mechanism comprises: an attachment plate configured to fix and attach the image display device; a frame to which the rotation mechanism is attached so that the attachment plate is upwardly or downwardly slidable with respect to the frame; a lock/unlock mechanism which locks or unlocks the attachment plate to or from the frame in a case where the image display device is positioned at the uppermost end position or a lowermost end position in the movement region of the rising/lowering with respect to the rotation mechanism; and a guide mechanism which guide the attachment plate so that the attachment plate is upwardly or downwardly slidable with respect to the frame.

3. The ultrasonic diagnosis apparatus according to claim 2, wherein the rotation mechanism comprises: a pair of first rotary blocks fixed and attached to the frame; a pair of second rotary blocks which are rotatable with respect to the first rotary blocks and have a substantially cylindrical outer peripheral surface; and a third rotary block which is rotatable with respect to the second rotary blocks, and
the second rotary blocks are disposed symmetrically on right and left sides of a rotary shaft of the third rotary block, and the first rotary blocks are disposed symmetrically on the right and left sides of the rotary shaft of the third rotary block so that the second rotary blocks are sandwiched between the first rotary blocks.

4. The ultrasonic diagnosis apparatus according to claim 3, wherein the rotation block mechanism comprises control blocks attached to the attachment plate, stoppers, engagement portions, and stop grooves,
the first rotary blocks are provided on outer peripheries thereof with first cutout portions extending in an axial direction,
the second rotary blocks are provided on outer peripheries thereof with second cutout portions extending in an axial direction and the stop grooves,
the first cutout portions constitute the engagement portions together with the second cutout portions,
the control blocks engage with the engagement portions so as to pass through block through holes disposed in the frame, so that the first rotary blocks and the second rotary blocks are integrally rotated, thereby limiting the rotation of the image display device to a horizontally reclined position when the device is positioned at a lowermost end with respect to the rotation mechanism, and
the stopper are configured to be inserted into the stop grooves, the stopper being configured to engage with the stop grooves so as to pass through the block through holes disposed in the frame, thereby limiting the rotation of the image display device to the horizontally reclined position during relative movement.

5. The ultrasonic diagnosis apparatus according to claim 4, wherein the stop grooves are formed by cutting a region constituted of a partial circle extending along an outer peripheral surface and a chord connecting both ends of the partial circle, and a cross section of the stop grooves in a radial direction is U-shaped.

6. The ultrasonic diagnosis apparatus according to claim 4, wherein the rotation mechanism further comprises a rotary range limiting section,
the rotary range limiting section comprises:
a first rotary range limiting section including guide pins or circular guide grooves disposed in the first rotary blocks, and circular guide grooves or guide pins correspondingly disposed in the surface of the second rotary blocks facing the first rotary blocks so as to correspond to the guide pins or the guide grooves of the first rotary blocks; and
a second rotary range limiting section including guide pins or circular guide grooves disposed in the other surfaces of the second rotary blocks, and circular guide grooves or guide pins correspondingly disposed in the surfaces of the third rotary block facing the second rotary blocks so as to correspond to the guide pins or the guide grooves of the second rotary blocks, and
the rotary range limiting section limits a rotary range of the image display device in the upward-downward direction in accordance with a length of the circular guide groove, limits the rotary range in accordance with the length of the circular guide groove in the second rotary range limiting section when the first rotary blocks and the second rotary blocks integrally rotate, and limits the rotary range in accordance with the length of the circular guide groove in the first rotary range limiting section when the second rotary blocks and the third rotary block integrally rotate.

7. The ultrasonic diagnosis apparatus according to claim 6, wherein the image display device is configured to be rotated in the range limited by the second rotary range limiting section when the image display device is disposed at the lowermost end position with respect to the rotation mechanism, and the image display device is configured to be rotated in the range limited by the first rotary range limiting section when the image display device is disposed at the uppermost end position with respect to the rotation mechanism.

8. The ultrasonic diagnosis apparatus according to claim 7, wherein the maximum value of a downward rotary angle limited by the first rotary range limiting section is 90°.

9. The ultrasonic diagnosis apparatus according to claim 4, wherein the first rotary blocks are further provided with attachment surfaces which are parallel with the axial direction and are fixed and attached to the frame, and
the rotary shaft is provided along an axial center of the third rotary block so as to extend to both the sides thereof, and the third rotary block is fixed and connected to the support arm at the center of the outer peripheral surface of the third rotary block in the axial direction.

10. The ultrasonic diagnosis apparatus according to claim 2, wherein the guide mechanism comprises guide rollers disposed on the attachment plate, and guide grooves disposed in the frame so as to correspond to the guide rollers 24.

11. The ultrasonic diagnosis apparatus according to claim 10, wherein the lock/unlock mechanism comprises two upper and lower lock holes disposed in the frame, a lock lever rotatably connected to the attachment plate via a link and engaged with the lock hole to lock the lock lever, and an urging spring,
positions of the lock holes 32 are set so that the lock lever is urged by the urging spring to be engaged with and locked in the upper or lower lock hole when the image display device is disposed at the uppermost end position or the lowermost end position with respect to the rotation mechanism, and
the lock lever is operated against an urging force of the urging spring to unlock the lock lever when the image display device is upwardly or downwardly moved.

12. The ultrasonic diagnosis apparatus according to claim 2, wherein the guide mechanism comprises guide plates fixed to the control blocks of the attachment plate so as to sandwich the frame therebetween, and a pair of rails disposed on the frame so that the guide plates are guided to coincide with a pair of through holes disposed in the guide plates.

13. The ultrasonic diagnosis apparatus according to claim 12, wherein the lock/unlock mechanism comprises two upper and lower positioning holes disposed in the side surface of each of the guide plates, a traction rod slidably disposed on a fixing block fixed to the frame, and an urging spring,
positions of the positioning holes are set so that the traction rod is urged by the urging spring to be engaged with and locked in the upper or lower positioning hole when the image display device is disposed at the uppermost end position or the lowermost end position with respect to the rotation mechanism, and
the traction rod is operated against the urging force of the urging spring to unlock the locked traction rod when the image display device is upwardly or downwardly moved.

14. The ultrasonic diagnosis apparatus according to claim 9, wherein the rotary shaft of the third rotary block is provided with disc springs disposed between the second rotary blocks and the third rotary block to increase a frictional force therebetween.

15. The ultrasonic diagnosis apparatus according to claim 2, wherein the support mechanism main body further comprises a mechanism which offsets or decreases a weight of the image display device.

16. The ultrasonic diagnosis apparatus according to claim 15, wherein the mechanism which offsets or decreases the weight of the image display device is tensile springs, each of which has one end connected to the attachment plate and the other end connected to the frame.

17. The ultrasonic diagnosis apparatus according to claim 15, wherein the mechanism which offsets or decreases the weight of the image display device is Conston springs, each of which has one end connected to the attachment plate and the other end connected to the frame.

18. An ultrasonic diagnosis apparatus comprising: an image display device, an ultrasonic probe, an operation panel, an apparatus main body and a support mechanism,
wherein the support mechanism comprises a support mechanism main body and a support arm,
the support mechanism main body comprises an attachment plate to fix and attach the image display device, an upper rotary shaft and a lower rotary shaft which constitute a rotary shaft to rotate the image display device in an upward-downward direction and which are attached to the attachment plate, and a shaft connecting portion made of an elastic material,
the shaft connecting portion comprises two upper and lower shaft attachment washers which are fixed and connected to the support arm and to which the upper and lower rotary shafts are attached, and
the lower rotary shaft is configured to rotate the image display device to a horizontal position.

19. The ultrasonic diagnosis apparatus according to claim 1, wherein the image display device further comprises a lowering speed suppression mechanism which suppresses a lowering speed during lowering of the image display device so that the lowering speed is lower than a free speed at which the image display device lowers owing to its own weight.

20. The ultrasonic diagnosis apparatus according to claim 19, wherein the lowering speed suppression mechanism comprises an image display device side fixing member fixed to the image display device in such a manner as to integrally upwardly or downwardly move together with the image display device, and
a support mechanism side urging member which is fixed to the support mechanism via a supporting point and which is switchable between a suppression state where the image display device side fixing member is upwardly urged and a release state where the image display device side fixing member is not urged,
when the image display device rises, the support mechanism side urging member is in the release state, and
when the image display device lowers, the support mechanism side urging member is in the suppression state, and the lowering speed of the image display device is set to be lower than a lowering speed at which the image display device lowers owing to its own weight.

21. The ultrasonic diagnosis apparatus according to claim 20, wherein the support mechanism side urging member is rotatably supported on the support mechanism via the supporting point.

22. The ultrasonic diagnosis apparatus according to claim 21, wherein the image display device side fixing member comprises a rack extending in the rising/lowering movement direction, and a cam having a guide groove,
the support mechanism side urging member comprises one end having a pinion which is damply rotated around an axial center to engage with the rack, and the other end having a pulley guided along the guide groove of the cam, the supporting point being positioned between the pinion and the pulley,
when the image display device rises, the pulley is guided along the guide groove to bring the support mechanism side urging member into the release state where the pinion is away from the rack, and
when the image display device lowers, the pulley is guided along the guide groove to bring the support mechanism side urging member into the suppression state where the pinion engages with the rack.

23. The ultrasonic diagnosis apparatus according to claim 22, wherein the rack and the cam are disposed on mutually deviated planes, the pinion is disposed on the plane where the rack exists, the pulley is disposed on the plane where the cam exists, and the support mechanism side urging member is disposed between the two planes.

24. The ultrasonic diagnosis apparatus according to claim 20, wherein the image display device side fixing member is a guide rail extending in the rising/lowering movement direction, and
the support mechanism side urging member comprises:
an electromagnetic coil stored in a coil frame which is the supporting point, and
a post as a core which comes in contact with the guide rail to apply an upward frictional force thereto when the electromagnetic coil is excited, and which is away from the guide rail when the excitation is released.

25. The ultrasonic diagnosis apparatus according to claim 24, wherein a portion of the post which comes in contact with or is away from the guide rail is elastically deformable.

26. The ultrasonic diagnosis apparatus according to claim 19, wherein the lowering speed suppression mechanism comprises:
an image display device side fixing member fixed to the image display device in a configuration of upwardly or downwardly moving integrally with the image display device, and
a support mechanism side urging member which is fixed to the support mechanism via a supporting point and which is switchable between a suppression state where the image display device side fixing member is upwardly urged and a release state where the image display device side fixing member is urged with a force smaller than the upward urging force,
when the image display device rises, the support mechanism side urging member is in the release state, and
when the image display device lowers, the support mechanism side urging member has the suppression state, and sets the lowering speed of the image display device to be lower than a lowering speed at which the image display device lowers owing to its own weight.

27. The ultrasonic diagnosis apparatus according to claim 26, wherein the support mechanism side urging member is rotatably fixed to the support mechanism via the supporting point.

28. The ultrasonic diagnosis apparatus according to claim 26, wherein the image display device side fixing member is a guide rail extending in the rising/lowering movement direction,
the support mechanism side urging member comprises two levers forming a roof-like shape which makes it possible to sandwich the guide rail between the levers, and
inwardly facing ends of the two levers come in contact with the guide rails, respectively, and outwardly facing ends of the two levers are the supporting points.

29. The ultrasonic diagnosis apparatus according to claim 28, wherein the inwardly facing ends of the two levers are elastically deformable.
